(19) United States
(12) Patent Application Publication
(10) Pub. No.: US 2006/0204526 A1
(43) Pub. Date: Sep. 14, 2006

(54) Emulsive composition containing Dapsone

(76) Inventors: Robert William Lathrop, Fort Collins, CO (US); David W. Osborne, Santa Rosa, CA (US)

Correspondence Address: Schwegman, Lundberg, Woessner & Kluth, P.A., P.O. Box 2938, Minneapolis, MN, 55402, US

(21) Appl. No.: 11/352,935

(22) Filed: Feb. 13, 2006

Related U.S. Application Data
(63) Continuation of application No. PCT/US04/26447, filed on Aug. 13, 2004.
(60) Provisional application No. 60/494,912, filed on Aug. 13, 2003.

(30) Foreign Application Priority Data
Feb. 24, 2005 (WO) 2005/016296
Aug. 13, 2004 (WO) PCT/US04/26351

(51) Int. Cl. A61K 9/00; A61K 31/135

(52) U.S. Cl. 424/400; 514/646

(57) ABSTRACT

The present invention relates to a topical, emulsive composition containing Dapsone or its derivative. The inventive composition incorporates emollients and Dapsone or its derivative in a stable emulsion. The stability is achieved through the use of a combination of certain surfactant mixtures and an enhancer providing solubility of the Dapsone.

### STATEMENT OF PRIORITY

This application is a Continuation Under 35 U.S.C. § 1.111(a) of International Application No. PCT/US2004/026447, filed Aug. 13, 2004 and published in English as WO 2005/016296 on Feb. 24, 2005, which claims the benefit under 35 U.S.C. § 119(e) of U.S. Provisional Application No. 60/494,912 filed Aug. 13, 2003, which applications are incorporated herein by reference.

### BACKROUND OF THE INVENTION

DDS or 4,4′-diaminodiphenyl sulfone has the USP name, Dapsone, and is a well-known medicament possessing several beneficial medicinal activities. Dapsone is typically administered as one of the medicinal agents used in the treatment of leprosy. Dapsone and its derivatives are also effective for treatment of bacterial infections, protozoanal infections such as malaria, pneumocystis carinii, and plasmonic infections such as toxoplasmosis. Some of the early publications describing Dapsone and its derivatives include a 1938 French patent (FR829,926) and U.S. Pat. No. 2,385,899. These references explain that Dapsone has an inhibiting effect on the growth of bacteria, mycobacteria, an plasmodia. According to the 2001 Physicians Desk Reference, Dapsone is commercially available in tablet form from Jacobus Pharmaceutical Company. Dapsone also used as a cross-linking agent for epoxy resins.

Dapsone is also known to be useful as an anti-inflammatory agent. It has been used to treat skin diseases characterized by the abnormal infiltration of neutrophils, such as Dermatitis herpetiformis, linear IgA dermatosis, pustular psoriasis, pyoderma gangrenosum, acne vulgaris, and Sweet's Syndrome.

In all of these applications including topical applications, Dapsone treatment is systemic and the drug is administered orally. No topical formulation of Dapsone is commercially available for local treatment of skin disease and references describing topical administration of Dapsone are not common. Of the few scientists considering topical administration, Osborne (U.S. Pat. Nos. 5,863,560 and 6,060,085) is one providing a topical formulation of Dapsone. He describes a dermatological gel composition containing Dapsone.

One reason for the lack of commercial topical formulations rests upon the solubility character of Dapsone and its derivatives. Dapsone and many of its derivatives are soluble in ethanol, methanol, acetone and dilute, aqueous HCl but are practically insoluble in water and in oils such as petroleum gel, wax and vegetable oils. Consequently, topical formulations of Dapsone in water or oils are difficult to develop. Those topical formulations of Dapsone that have been developed typically include salt formers and solubilizing agents that enable formation of a single phase aqueous solution or gel. The solubilizing agents are water miscible and include such organic liquids as ethylene diglycol monoethyl ether and ethanol.

However, use of such topical formulations of Dapsone is also problematic. These topical formulations typically act as drying agents for the skin. They remove essential oils and natural skin softeners from the skin thus causing it to be dry, itch and crack. Inclusion of exogeneous skin emollients, oils and the like, however, causes phase separation and precipitation of Dapsone. Use of typical emulsifiers does not solve the Dapsone precipitation owing to the lowered Dapsone solubility and conflicting physical characteristics of the phases of the resulting composition.

Therefore, there is a need to formulate a stable, aqueous based, emulsive Dapsone composition that will not dry or crack the skin. There is a further need to formulate such a composition with pharmaceutically acceptable ingredients. There is also a need to include exogeneous oils, emollients and the like in such an emulsion without causing separation or precipitation of the Dapsone.

### SUMMARY OF THE INVENTION

These and other needs are achieved by the present invention which provides a stable, emulsive composition containing Dapsone or a derivative thereof. The emulsive composition enables the use of a wide variety of oil phase components as vehicles for the topical (skin or mucosa) delivery of Dapsone or a derivative thereof. The emulsive composition of the invention also provides for the use of polar phase components for the augmented delivery and enhancement of Dapsone or a derivative thereof on the skin or mucosa.

Accordingly, the present invention is directed to an emulsive composition of the following components: a) Dapsone or its derivative (hereinafter collectively termed Dapsone), b) a solvation medium (polar phase component) for Dapsone c) an emulsifier system, d) an oil phase component, e) optional water and f) optional gelation or thickening agents. Excipients as well as other additives and colorants may also be included as additional compounds in the solvation medium (polar phase) and oil phase components. Each of the components of the emulsive composition (except the optional water) can be composed of one or more individual compounds falling within the component description.

The solvation medium (polar phase) may be an organic solvent that ranges in water solubility from moderately soluble (for example having from 2% to 10% by weight solubility in water) to completely miscible in water in all proportions. The solvation medium will at least partially, and preferably will completely dissolve Dapsone. When optional water is combined with the solvation medium, the combination also at least partially, and preferably completely, dissolves the Dapsone. In either aspect, the solvation medium or solvation medium plus water dissolves or disperses the Dapsone as a stable solution or dispersion. When the combination of solvation medium and water are employed, that combination is the polar phase (an aqueous polar phase) and the solvation medium preferably enhances the solubility of the Dapsone in this aqueous polar phase. Preferred organic solvents that function as the solvation medium either alone or in combination with water include a polyglycol, a polyol, a polyglycol ether, a polyol ether, a polyglycol monoether or a polyol monoether or a combination thereof.

The oil phase component includes any pharmaceutically acceptable organic, hydrophobic substance that softens and moistens the skin layers such as the epidermis and dermis. Waxes, oils, fatty acids, polyols, and esterified fatty acids are some examples of the oil phase component.

The emulsifier system has both ionic and nonionic properties so that it stabilizes the emulsive composition of the invention and prevents Dapsone separation. Preferably, the ionic properties are anionic properties. The combination of these properties can be achieved by a mixture of surfactant and a saturated and/or unsaturated fatty alcohol. In particular, a blend of a C10 to C24 saturated and/or unsaturated fatty alcohol, and any one of more of a C8 to C24 saturated and/or unsaturated fatty alcohol phosphate ester or diester, a C8 to C24 saturated and/or unsaturated fatty alcohol sulfate ester or diester, a C8 to C24 saturated and/or unsaturated fatty alcohol carbonate ester or diester as well as derivatives of such saturated and/or unsaturated fatty alcohol phosphate, sulfate and/or carbonate esters may serve as the emulsifier system according to the invention. Preferably, the emulsifier system is a combination of a C12 to C18 fatty alcohol, a phosphate diester of a C12 to C18 fatty alcohol and a phosphate monoester of an unsaturated C12 to C18 fatty alcohol.

According to the invention, the concentrations of the components by weight relative to the total weight of the emulsive composition are as follows:
a) Dapsone may range from about 0.005 percent to about 30 percent, preferably about 0.1 percent to about 25 percent, more preferably about 0.1 percent to about 15 percent, especially more preferably about 0.1 percent to about 10 percent, very especially more preferably about 0.2 percent to about 8 percent, and most preferably about 0.5 to about 5 percent by weight of the emulsive composition, with such percentages as 1, 2, 5 and 7.5 being especially preferred embodiments thereof;
b) The solvation medium may range from about 0.5 percent to about 99 percent, preferably about 0.5 percent to about 50 percent, more preferably about 5 percent to about 40 percent, especially more preferably about 5 percent to about 35 percent, most preferably about 5 percent to about 30 percent;
c) The emulsifier system may range from about 0.1 percent to about 30 percent, preferably about 0.5 percent to about 25 percent, more preferably about 1 percent to about 25 percent, most preferably about 5 percent to about 25 percent, most preferably about 5 percent to about 20 percent;
d) The oil phase may range from about 0.1 weight percent to about 75 percent, preferably about 0.1 to about 50 percent, more preferably about 1 to about 45 percent, most preferably about 2 to about 40 percent;
e) Water may range from 0 percent to about 99 percent, preferably from 0 to about 50 percent, more preferably from 0 to about 40 percent, most preferably from 0 to about 35 percent, i.e., water is optional;
f) The amounts are combined to equal 100 percent, and except for water, each of the components a-d is to be included. Each of the four ingredient components a-d may be composed of one or more individual compounds falling within the designated component category.

The emulsive composition of the invention provides therapeutic benefits such as, but not limited to, anti-inflammatory activity, antibacterial activity, anti-itch activity and emollient properties so that it is useful in the treatment of such dermatological disorders as psoriasis, dermatitis and the itch associated with healing or gealed burn wounds while maintaining skin and/or mucosal integrity, flexibility, stretch and moisturization.

### Definitions

As used herein, certain terms have the following meanings. All other terms and phrases used in this specification have their ordinary meanings as one of skill would understand. Such ordinary meanings may be obtained by reference to such technical dictionaries as Hawley's Condensed Chemical Dictionary 11^{th} Edition, by Sax and Lewis, Van Nostrand Reinhold, New York, N.Y., 1987; The Merck Index, 11^{th} Edition, Merck & Co., Rahway N.J. 1989; The Physician's Desk Reference (PDR), 2001 Edition, Medical Economics Company, Montvale, N.J.; Stedman's Medical Dictionary, 25^{th} Edition, Williams & Wilkens, Baltimore, Md., 1990.

Dapsone is 4,4′-diaminodiphenyl sulfone. It has the chemical formula C₁₂H₁₂N₂O₂S and is alternatively known as 4,4′-sulfonyldianiline or bis (4-aminophenyl)sulfone (also spelled sulphone). See the above-referenced Merck Index at entry no. 2820.

Derivatives of Dapsone refer to compounds that have a similar chemical structure and thus similar therapeutic potential to Dapsone. These include compounds with two organic substituents (R₁, R₂) at the two amino groups (R₁R₂NC₆H₄SO₂C₆ H₄NR₁R₂). R₁ and/or R₂ each may be hydrogen, C₁ to C₆ alkyl, C₁ to C₆ alkoxyoyl as well as a substituted alkyl group of 1 to 6 carbons wherein the substituent may be hydroxyl, thio, alkoxy, halo, amido and similar polar or lipophilic substituents. Preferably, R₁ and R₂ are the same. When the R₁ and R₂ substitution is R═CHO, the compound formed is generically named diformylDapsone. It is alternatively known as bis (4-formaminophenyl)sulfone and 4,4′-diformyldiaminodiphenyl sulfone. When the R₁ and R₂ substitution is R═COCH3, the compound formed is AceDapsone, alternatively named bis (4-acetamidophenyl)sulfone and 4,4′-diacetyldiaminodiphenyl sulfone. AceDapsone is a known prodrug of Dapsone. Other derivatives known to have antibacterial and/or anti-inflammatory effect are glucosulfone sodium, solapsone, diathymosulfone, acediasulfone, monoacetyl Dapsone, acetosulfone, succisulfone, aldesulfone sodium, and thiazolsulfone. Additional Dapsone derivatives are described in the following journal articles, the disclosures of which are incorporated herein by reference: M. D. Colman et al. J. Pharm. Pharmacol., 1997, 49, 53-57; J. Pharm. Phamracol., 1996, 48, 945-50; Environmental Toxicology and Pharmacology, 1996, 2, 389-395.

An emulsifying agent is a surfactant (defined separately below). However, not all surfactants are emulsifying agents. An emulsifying agent is typically a term used to describe an organic compound that stabilizes a uniform dispersion of one solvent in another where the two solvents are immiscible. Portions of the emulsifying agent dissolve in the different phases so that the dispersion is prevented from coalescing into two separate liquids.

A fatty alcohol is a saturated or unsaturated C8 to C40 alcohol that may or may not be substituted by additional groups such as halo, alkoxy of 1 to 6 carbons, alkyl keto of 2 to 6 carbons, alkoxycarbonyl of 2 to 6 carbons, alkyl amido of 2 to 6 carbons and alkye amine of 1 to 6 carbons optionally substituted with 1 or 2 alkyl groups of 1 to 4 carbons on the amine.

The terms “insoluble” and “immiscible”, as applied to two liquids, mean that one liquid displays essentially no solubility in the second. While the measurable solubility need not be zero, for the practical purposes of formulating topical products, the level of solubility is insignificant if an ingredient is described as insoluble or immiscible in another.

The term “miscible” when used in connection with two liquids means that the two liquids are soluble in each other at all ratios.

A solution is a system at chemical equilibrium in which a solute (liquid, solid, or gas) is dissolved in a liquid solvent.

A surfactant or surface active agent is an organic compound that reduces the surface tension when dissolved in water or water solutions. In an emulsion, a surfactant will contain a hydrophilic portion and a lipophilic portion by which it functions to reduce the surface tension of the surfaces between immiscible phases. Functionally, in dermatological applications, surfactants include emulsifying agents, wetting agents, cleansing agents, foam boosters, and solubilizing agents. A surfactant is any nonionic, anionic, or cationic organic compound of moderate to high molecular weight (such as from about 100 to 300,000 daltons) for which a significant portion of the molecule is hydrophilic and a significant portion is lipophilic.

The term “pharmaceutically active agent” is used to refer to a chemical material or compound that is suitable for topical administration and induces a desired physiological effect.

The term “topical administration” means the delivery of a composition or active agent to the skin or to mucosal tissue. A topical composition is one that is suitable for topical administration.

The term “about” means a variation of 10 percent of the value specified; for example about 50 percent carries a variation from 45 to 55 percent.

### DETAILED DESCRIPTION OF THE INVENTION

The present invention solves the formulation and treatment problems associated with topical administration of Dapsone and its derivatives (hereinafter collectively termed Dapsone). These compounds are aromatic, are substituted with diamino groups and are difficult to formulate as aqueous based topical compositions. The compounds themselves readily separate and/or precipitate from such aqueous based compositions. When solvation enhancers are used, the resulting compositions typically cannot include desirable, oil-based skin conditioning agents. Such skin conditioning agents, however, are common formulation ingredients for topical compositions because without them, topical compositions often dry, redden and are detrimental to the skin.

According to the invention, it has been discovered that combinations of solvation medium ingredients and emulsifiers enable the formulation of Dapsone topical compositions that include oil-based skin conditioning agents. In particular, the emulsive composition of the invention includes Dapsone, a solvation medium, an emulsifier system and one or more oil-based skin conditioning agents. An alternative emulsive composition of the invention includes water with the solvation medium so as to provide an aqueous polar phase.

The emulsive composition of the present invention may display a consistency and feel characteristic of products suitable to application to the skin or a mucous membrane. The consistency of the composition may be a freely-flowing liquid. Such a consistency allows for a rapid spreading on the skin and an ease of application. Alternately, the consistency of the composition may range to a stiff or firm, semi-solid. A stiff consistency may be suitable for a heavier application of the composition to a limited site on the skin or on a mucous membrane. Further, a stiff consistency resulting from a high oil phase may contribute to the occlusive property of the composition on the skin or a mucous membrane. The feel of the composition on the skin may range from a thin, wet feel to a stiff, waxy feel. With the adjustment of the various ingredients the composition can be formulated to display a consistency and feel optimal for the delivery of the Dapsone for an intended indication.

### Composition of the Invention

Many dermatological products are described as emulsions but the two immiscible phases forming such products often do not form colloidal mixtures. Instead, the internal phases are dispersed as droplets within the continuous phases to create temporarily stable systems. The chemical equilibria in such systems are toward the separation of the immiscible phases.

A system may be said to be at chemical equilibrium when it is stable theoretically forever as a result of random molecular movement. In contrast, a physically stable topical emulsion system often involves a practical and limited stability. An emulsion may be classified as physically stable when it displays no or insignificant change in the phase dispersion over a defined period of time. For a dermatological emulsion product, a physically stable system typically is a system that shows no or insignificant change in the phase dispersion over the period of a marketable self-life.

In dermatological or topical products, common emulsions are oil-in-water emulsions and water-in-oil emulsions. In the former, the oil phase is the internal phase dispersed in the continuous water phase. In the latter, the oil phase may be the continuous phase. More complex emulsion systems have been described and formulated as dermatological products. Water-in-oil-in-water emulsions and other complex combinations may be formed between immiscible phases.

In many topical emulsions, an internal oil phase contains oily or fatty excipients that are solid at room temperature, thereby raising a point of confusion over the definition of an emulsion as a liquid-in-liquid dispersion. This point is clarified by the understanding that at the time of formation, the emulsion is a liquid-in-liquid dispersion because the oil phase may have been heated or otherwise manipulated by make it a liquid. It may also be noted that at the water/oil interface the precise nature of the physical state of the oil phase as either a liquid or a solid is not a simple characterization.

The oil phase of a topical emulsion may contain oily or fatty materials that are miscible or compatible with each other but that have no or insignificant miscibility or solubility in water. As many oil phase excipients are solids at standard temperature, the miscibility is commonly evaluated with the excipients in their liquid states.

In topical or dermatological products, the water phase, or aqueous phase, often contains an amount of water and optionally a variety of liquids or solids that are soluble, miscible, or dispersed in the water.

Many of these properties are present in the emulsive composition of the present invention. However, water need not be present in combination with the solvation medium according to the invention.

In the following discussion, use of the term “Dapsone” shall mean Dapsone or its derivative unless otherwise stated.

The present invention provides a physically stable emulsive composition containing Dapsone in a solvation medium (polar phase) in combination with at least one oil phase component (oil phase) and an emulsifying system. The solvation medium (polar phase) includes an organic solvent for solvating the Dapsone. Optionally, the solvation medium may contain additional compounds such as common excipients, coloring agents and the like. Also optionally, the solvation medium may form a combination with water to act as the polar phase.

The emulsifying system may be a combination of a fatty alcohol and a surfactant.

The emulsive composition can be formulated into a range of topical compositions, from light, non-greasy lotions to heavy, emollient creams.

According to the invention, the concentration of Dapsone may be any amount that provides effective antibacterial and/or anti-inflammatory properties to the emulsive composition. In particular, the concentration of Dapsone in the emulsive composition of the invention may range from about 0.05 percent to about 30 percent by weight of the emulsion formulation. Preferably, this concentration may be from about 0.1 percent to about 25 percent, more preferably about 0.1 percent to about 15 percent, especially more preferably about 0.1 percent to about 10 percent, very especially more preferably about 0.2 percent to about 8 percent, and most preferably about 0.5 to about 5 percent by weight of the emulsive composition. The Dapsone concentration of especially preferred embodiments may be such percentages as 1, 2, 5 and 7.5.

According to the invention, the solvation medium may be an organic solvent that is moderately soluble to miscible with water and dissolves Dapsone or enables dissolution of Dapsone in the combination of solvation medium and optional water. The solvation medium or its combination with water acts as the polar phase of the emulsive composition.

Preferably, in either alternative, namely, use of an organic solvent or solvents alone as the solvation medium or use of the combination of the organic solvent or solvents and the water enables the complete dissolution of Dapsone in the emulsive composition. However, the amount of the organic solvent used alone as the solvation medium or the concentration of organic solvent in the combination of water and solvation medium may also enable partial dissolution of the Dapsone in the emulsive composition. In the latter situation, the portion of Dapsone not dissolved in the solvation medium or combination may be suspended as a dispersion of microparticles or micronized particles and the like in the emulsive composition. Alternatively, the portion of Dapsone not dissolved may be suspended as a dispersion of crystalline Dapsone. The size of the suspended particles of Dapsone may be controlled by the preparation of the Dapsone raw material or by the process by which the emulsive composition is compounded. The size of the suspended particles may range from below 10 microns (microparticles or micronized particles) to palpable particles above about 100 microns. The emulsifying system participates in the maintenance of this dispersion. Alternatively, the undissolved portion of Dapsone may be dissolved in the oil phase of the emulsive composition when it is formed by combination of the solvation medium, the oil phase and the emulsifying system.

Partial dissolution of Dapsone may be the result of any one or more of a number of formulation designs. First, the organic solvent may not enable complete dissolution of the desired concentration of Dapsone in the solvation medium even though lower amounts of Dapsone will be completely dissolved. Second, the volume of the oil phase may be insufficient to dissolve this portion of Dapsone not dissolved in the solvation medium. Third, the formation of the emulsive composition may decrease the solubility of Dapsone in the solvation medium because of interaction of the oil phase, the emulsifying system and the solvation medium.

Notwithstanding the dissolution characteristics of Dapsone in the salvation medium and in the emulsive composition, in a preferred embodiment of the invention, the amounts of Dapsone and organic solvent are selected to fully dissolve Dapsone in the neet organic solvent. Although the dissolution of Dapsone in organic solvent may be complete, subsequent formation of the emulsive composition may result in partial precipitation of Dapsone or maintain complete dissolution of Dapsone. Both possibilities are within the invention.

According to the invention, the concentration of the solvation medium as the organic solvent alone relative to the total weight of the emulsive composition ranges from about 0.5 percent to about 99 percent by weight. More preferably the concentration of solvation medium is from about 0.5 percent to about 50 percent by weight. Especially more preferably the concentration of solvation medium is from about 5 percent to about 40 percent, very especially more preferably about 5 percent to about 35 percent by weight, and most preferably about 5 percent to about 30 percent by weight of the emulsion composition.

When water is combined with an organic solvent or solvents as the solvation medium, the concentration of solvation medium relative to the weight of the water plus solvation medium ranges from 0.005 weight percent to 98 weight percent. The ingredients in this instance are the organic solvent or solvents and water.

The concentration of the organic solvent in the emulsion will vary depending on the desired Dapsone concentration, the solubility of Dapsone in the solvation medium, and the desired extent to which the Dapsone is dissolved in the emulsive composition. Dapsone solubility in some organic solvents exceeds thirty percent by weight of the solution. Its solubility in other organic solvents can be less than one percent by weight. Suitable emulsive compositions can be formulated with an organic solvent calculated to dissolve an effective amount of the Dapsone. Further, the concentration and ratio of two or more organic solvents may be selected for optimal effect depending upon a synergistic solubility of Dapsone.

Organic solvents that are suitable for use as the solvation medium in the present invention and are moderately soluble to miscible with water, can be classified into a number of broad groups. One group is glycol ethers. A glycol ether is an ether formed from at least one glycol and at least one lower alkyl alcohol. Preferably the glycol is selected from an alkylene glycol such as ethylene glycol, propylene glycol, or butylene glycol. The ether portion of the glycol ether is a radical of a lower alkyl alcohol such as a C1 to C6 alcohol. Preferably, the ether portion alcohol is selected from methyl alcohol, ethyl alcohol, propyl alcohol, isopropyl alcohol, butyl alcohol, or isobutyl alcohol. The glycol ethers have a generalized formula of CₓH_{y}O_{z} where x is from 4 to 10, y is from about 10 to 22, and z is from 2 to 5. According to the present invention, the glycol ethers are soluble or miscible with water and range in molecular formula from C₄ to about C₁₀.

Examples of glycol ethers under the classification of ethylene glycol ethers include ethylene glycol monopropyl ether (propoxyethanol), ethylene glycol monobutyl ether (butoxyethanol), diethylene glycol monomethyl ether (methoxydiglycol), diethylene glycol monoethyl ether (ethoxydiglycol), diethylene glycol monobutyl ether (butoxydiglycol), diethylene glycol monoisopropyl ether (isopropyldiglycol), and diethylene glycol monoisobutyl ether (isobutyl diglycol).

Glycol ethers under the classification of propylene glycol ethers include propylene glycol monomethyl ether, dipropylene glycol monomethyl ether (PPG-2 methyl ether), tripropylene glycol monomethyl ether (PPG-3 methyl ether), propylene glycol n-propyl ether, dipropylene glycol n-propyl ether (PPG-2 propyl ether), propylene glycol monobutyl ether, dipropylene glycol monobutyl ether (PPG-2 butyl ether), propylene glycol monoisobutyl ether, and dipropylene glycol dimethyl ether. In one embodiment of the invention the solvation enhancer is ethoxydiglycol. In another embodiment, the solvation enhancer is butoxydiglycol.

A second group of organic solvents useful in the present invention includes the compounds classified as diols. A diol is an organic compound with two hydroxyl groups. It will be understood that an ether glycol as presented above may contain two hydroxyl groups and may therefore be classified as a diol. Diols suitable for use in the present invention include diethylene glycol, triethylene glycol, propylene glycol, propanediol, dipropylene glycol, butylene glycol, hexylene glycol, pentylene glycol, and isopentyldiol.

Additional organic solvents suitable for use in the present invention that are moderately soluble to miscible in water include mono alcohols of the formula C1 to C10, and esters thereof including, but not limited to, dimethyl isosorbide, benzyl alcohol, triacetin, diacetin, ethanol, butyl alcohol, propylene carbonate, butylene carbonate, ethoxydiglycol acetate, 1-methyl-2-pyrrolidone, dimethylsulfoxide, ethoxydiglycol acetate, and isopropyl alcohol.

Another group of suitable organic solvents includes the polymers of ethylene oxide up to a molecular weight of approximately 700. Under the International Nomenclature of Cosmetic Ingredients classification these compounds are known as PEG-4 through PEG-16.

The oil phase component of the emulsive composition of the invention may include a general class of compounds that will dissolve Dapsone. Although these do not constitute solvation medium for Dapsone, they enable complete or further dissolution of Dapsone in the two phases of the emulsive composition. These compounds include liquids that are either not soluble in the organic solvent or the combination of organic solvent and water, or have insufficient solubility in the organic solvent or its combination with water at a concentration selected for use. Many of these compounds are oily liquids that can be combined with water and/or organic solvent to form an emulsion. When such a compound is selected as an oil phase component, it is selected for Dapsone solubility. It may also constitute the entire oil phase of the emulsion.

One broad grouping of such oil phase compounds for additional dissolution of Dapsone includes the di-esters formed between a dicarboxylic acid, e.g., oxalic acid, succinic acid, maleic acid, glutaric acid, adipic acid, sebacic acid, and an alkyl alcohol, e.g., isopropyl alcohol, isobutyl alcohol, butyl alcohol, ethyl alcohol, hexyl alcohol, isodecyl alcohol, isononyl alcohol, ethylhexyl alcohol, propyl alcohol. Common examples include diethyl sebacate, diisopropyl adipate, diisobutyl adipate, diisopropyl sebacate, diethyl succinate, and dipropyl adipate.

A second group of such oil phase compounds includes mono-esters formed between a monocarboxylic acid and an alkyl or aralkyl alcohol. Examples of the monoacids include palmitic acid, lauric acid, oleic acid, myristic acid, isostearic acid, linoleic acid, linolenic acid, ricinoleic acid and benzoic acid. Examples of the alkyl or aralkyl alcohol include isopropyl alcohol, ethyl alcohol, propyl alcohol, butyl alcohol, isobutyl alcohol, 2-etylhexyl alcohol, isodecyl alcohol or benzyl alcohol. Common examples include ethyl oleate, ethyl palmitate, isopropyl myristate, isopropyl palmitate, isobutyl palmitate, benzyl benzoate and octyl palmitate.

A number of these and similar ester compounds are supplied commercially by Croda (Oleochemicals) under the general trade name Crodamol and by Scher Chemicals under the general trade name Schercemol.

Additional compounds which may constitute the oil phase of the emulsion include, but are not limited to, oleic acid, oleyl alcohol, oleyl oleate, caprylic/capric triglyceride, propylene glycol dicaprylate/dicaprate, propylene glycol dilaurate, propylene glycol dipelargonate, myristyl mysistate, myristyl lactate, PPG-2 myristyl ether propionate, ethoxydiglycol oleate, octyldodecanol, bisabolol, and isostearic acid.

Of particular interest is the selection of combination of an organic solvent and an oil phase component wherein the two have at least some compatibility. This combination is illustrated by the compatibility shown between some water-soluble organic solvents and some water insoluble organic liquids. Many oil-phase compounds such as isopropyl myristate, isopropyl palmitate, and ethoxydiglycol oleate are immiscible with water but will form homogeneous solutions with water-soluble organic solvents. Ethoxydiglycol, butoxydiglycol, and dimethyl isosorbide, for example, are all water miscible liquids that will act as solvents for many oil-phase liquids in the absence of water to form homogenous mixtures.

According to a preferred embodiment of the invention, the above-described physical compatibility between the organic solvents constituting the solvation medium and the oil phase permits selection of formulations where organic liquids of the oil phase and the organic solvents of the polar phase of the emulsive composition can combine in the absence of water to form a homogeneous solvent mixture for Dapsone.

According to the invention, the emulsive composition includes a polar phase and an oil phase that can be rendered physically stable with the inclusion of an emulsifier system. According to the invention, the emulsifier system includes at least a fatty alcohol and a surfactant. This combination of a fatty alcohol and a surfactant may be self-emulsifying, and it may act as the emulsifier to disperse other fatty or oily compounds and the Dapsone into an emulsion with the solvation medium.

According to the invention, the surfactant portion of the emulsifier system includes non-ionic, anionic and cationic surfactants. Preferably, the surfactant portion of the emulsifier system is a non-ionic or anionic surfactant. Especially preferably, the surfactant portion of the emulsifier system is a non-ionic surfactant.

Non-ionic surfactants may include those from the following groups: polyoxyethylene sorbitan esters, e.g., polysorbate 20 and polysorbate 80; sorbitan esters, e.g., sorbitan stearate and sorbitan sesquioleate; polyoxyethylene glycol esters, e.g., PEG-4 dioleate and PEG-20 palmitate; polyoxyethylene ethers, e.g., ceteth-20, laureth-4, and steareth-10; polyoxyethylene alkoxylated alcohols, e.g., PEG-40 hydrogenated castor oil and PEG-5 lanolin; polyoxyethylene/polyoxypropylene block polymers, e.g., poloxamer 217 and poloxamer 237; polyoxyethylene phenol ethers, e.g., nonoxynol 10. Sulfate, phosphate and carbonate mono, di and tri esters of fatty alcohols are also included within the group of non-ionic surfactants.

Ionic surfactants suitable for use include the sodium and potassium salts of sulfated higher primary aliphatic alcohols. Examples include sodium caprylyl sulfonate, sodium cetyl sulfate, sodium cetearyl sulfate, sodium decyl sulfate, sodium lauryl sulfate, sodium myristyl sulfate, sodium oleyl sulfate, sodium octyl sulfate, sodium tridecyl sulfate, and potassium lauryl sulfate.

A second group of compatible anionic surfactants are those described as sodium salts of sulfated ethoxylated fatty alcohols. Examples include sodium deceth sulfate, sodium myreth sulfate, the sodium laureth sulfates, sodium laneth sulfate, and sodium trideceth sulfate. Another group of common anionic surfactants is the salts of the polyoxyethelene ether surfactants that form esters with phosphoric acid. Examples include sodium C13-15 pareth-8 butyl phosphate, sodium diceteareth-10 phosphate, sodium dioleth-8 phosphate, sodium oleth-7 phosphate, and sodium steareth-4 phosphate. Similar surfactant groups may be formed with the replacement of the phosphate by sulfate, carboxylate, or tartrate.

It will be understood that for all of the above anionic surfactants, a simple substitution of the cation, of the fatty alcohols, of ethoxylated chains, or of the complex anion make it possible to produce a huge array of similar surfactants. The foregoing is intended as an explication of possible agents; it is not meant as a definitive list or intended to limit the range of suitable surfactants for use in an emulsion system.

A third group of surfactants suitable for use as an emulsifying agent are cationic surfactants. A prominent group of cationic surfactants suitable for this function are formed from quaternary ammonium salts. Examples include behentrimonium chloride, behentrimonium methosulfate, benzalkonium chloride, cetrimonium chloride, cetrimonium methosulfate, dicetyldimonium chloride, distearyldimonium chloride, lapyrium chloride, lauralkonium chloride, stearalkonium chloride, and PEG-3 distearoylamidoethylmonium methosulfate, quaternium-24 (decyl dimethyl octyl ammonium chloride).

Suitable surfactants may be incorporated individually into the emulsifier system of the invention or used in combination of two or more to permit the development of an emulsifier system according to the invention.

According to the invention, a surfactant may be blended with a fatty alcohol to form the emulsifier system of the emulsive composition. Such blends may be synergistic combinations of at least one fatty alcohol and at least one surfactant. The surfactant may be anionic and/or non-ionic. The fatty alcohol/surfactant blend may be self-emulsifying, and it may also act as an emulsifying agent for other oil phase components.

A wide variety of commercial blends of fatty alcohol and surfactant are available. Croda, Inc. manufactures Emulsifying Wax N.F. under the trade names Polawax® and Polawax®A-31. Croda also supplies a series of blends of cetearyl alcohol and ceteareth-20 under the name Cosmowax®. Croda also manufactures an anionic self-emulsifying wax, Crodafos® CES, which is a blend of cetearyl alcohol, dicetyl phosphate, and ceteth-10 phosphate. Gattefosse also manufactures a number of suitable blends. Gattefosse's Emulcire 61® is a blend of cetyl alcohol, ceteth-20, and steareth-20.

According to the invention, a preferred range for the concentration of Crodafos® CES as the emulsifier system of the present invention is from 1 percent to 20 percent by weight, with a more preferred range of from 4 percent to 12 percent by weight. Similarly, a formulation useful according to the present invention is Gattefosse's Emulium Delta®, which is a blend of cetyl alcohol, glyceryl stearate, PEG-75 stearate, ceteth-20, and steareth-20. Preferred concentrations for Emulium Delta® are from about 3 percent to about 10 percent by weight.

In another embodiment of the present invention, the emulsifying system is selected from among the copolymers of acrylic acid crosslinked with allylpentaerythritol. The INCI designation for these emulsifying agents is acrylates/C10-30 alkyl acrylate crosspolymer. The National Formulary monograph for this material is under the name Carbomer Copolymer. These materials are marketed by Noveon, Inc. of Cleveland, Ohio under the trademarks Pemulen TR1 and Pemulen TR2. These agents may be used alone as the emulsifying system or they may be used in combination with a surfactant or surfactants to make up the emulsifying system of the invention.

While the oil phase component of the emulsion may be made up of a liquid organic compound that dissolves Dapsone, additional oil phase ingredients can be incorporated to provide a range of emulsion products. As is understood in the topical formulation art, these excipients may include various oils, waxes, emollients, thickening agents, occlusives, and skin-conditioning agents. Oil phase excipients may include cetyl alcohol, stearyl alcohol, cetyl palmitate, cetyl citrate, white wax, white petrolatum, paraffin, microcrystalline wax, stearyl citrate, ethoxydiglycol behenate, stearyl dimethicone, myristyl myristate, cetyl esters wax, dimethiconol stearate, octyl stearate, aluminum stearate, sodium stearate, ozokerite wax, shea butter, octyl stearate.

One of ordinary skill in the art will understand that a number of groups of excipients useful for topical formulations may be added to the emulsive composition of the invention. One such group of excipients suitable for addition to the water phase is water-soluble or water-dispersible gelling agents. Examples of such agents include the polyacrylic acid polymers, guar gum, polyquaternium-10, hyaluranic acid, sodium hyaluronate, xanthan gum, polyvinyl alcohol, hydroxyethylcellulose, xanthan gum, hydroxypropylmethylcellulose, and sodium carboxymetholcellulose.

Excipient groups well known in the formulation art may be added to augment the oil phase of the emulsive composition of the invention. These groups include antioxidants, represented by tocopherol, butylatedhydroxytoluene, butylatedhydroxyanisole, propyl gallate, tocopherol, tocopherol acetate, ascorbic acid, ascorbyl palmitate, and citric acid; and preservatives, represented by potassium sorbate, sorbic acid, benzoic acid, potassium benzoate, methylparaben, propylparaben, butylparaben, benzyl alcohol, dimethylol-dimethyl hydantoin, imidazolidinyl urea, diazolidinyl urea, and methylisothiazolinone.

Other groups of excipients useful for inclusion in the emulsive composition of the invention include buffering agents, neutralizing agents, humectants, chelating agents, colorants and opacifying agents, fragrances, skin conditioning agents, solubilizing agents such as the cyclodextrins, and biological additives.

The pH value of the composition may be adjusted with the addition of an acid or base, alone or in combination. Of particular value for the invention, a base may be added to neutralize the embodiments of the composition which contain a polyacrylic acid polymer or other acidic component. Such a polymer may be present as either a thickening or gelling agent or present as an emulsifier. Further, more than one polyacrylic acid polymer may be present in the composition. A base may be added to neutralize the composition to within a pH range to allow for the desired performance of the polyacrylic acid polymer. A suitable base may be selected from an inorganic base such as sodium hydroxide and potassium hydroxide, or it may be selected from an organic base such as diethanolamine, triethanolamine, and diisopropylamine. Likewise, an organic acid may be used to neutralize a basic component such as an amine containing surfactant.

The following examples are intended to further illustrate, but not limit, the invention.

### EXAMPLE 1

The following formulation was prepared:

| Excipient | % w/w |
|---|---|
| Dapsone | 5.0 |
| White petrolatum | 10.0 |
| Isopropyl palmitate | 5.0 |
| Crodafos ® CES¹ | 10.0 |
| Purified water | qs 100 |
| Ethoxydiglycol | 25.0 |
| Methylparaben | 0.2 |
| Propylparaben | 0.05 |

¹Crodafos ® CES is manufactured by Croda, Inc. It is a blend of cetearyl alcohol, dicetyl phosphate, and ceteth-10.

Procedure:
1) The white petrolatum, isopropyl palmitate, and Croda® CES were combined and melted at 70° C.
2) The methylparaben and propylparaben were dissolved in the ethoxydiglycol.
3) The Dapsone was dissolved in step 2) ethoxydiglycol solution.
4) The purified water was warmed to 70° C.
5) With high-speed mixing the oil phase of step 1 was added to the water phase of step 3. The emulsion was mixed to form uniform dispersion of oil phase.
6) The Dapsone solution was added to step 5) emulsion with continued mixing. The emulsion was cooled.

### EXAMPLE 2

The following formulation was prepared:

| Excipient | % w/w |
|---|---|
| Dapsone | 3.0 |
| White petrolatum | 5.0 |
| Isopropyl palmitate | 5.0 |
| Emulium Delta ®¹ | 10.0 |
| Purified water | qs 100 |
| Carbopol 980 | 0.25 |
| Ethoxydiglycol | 15.0 |
| Methylparaben | 0.2 |
| Propylparaben | 0.05 |
| Sodium hydroxide solution, 10% | 0.25 |

¹Emulium Delta ® is manufactured by Gattefosse. It is a blend of cetyl alcohol, glyceryl stearate, PEG-75 stearate, ceteth-20, and steareth-20.

Procedure:
1) The white petrolatum, isopropyl palmitate, and Emulium Delta® were combined and melted at 70° C.
2) The methylparaben and propylparaben were dissolved in the ethoxydiglycol.
3) The Dapsone was dissolved in step 2) ethoxydiglycol solution.
4) The Carbopol 980 was dispersed into the purified water with propeller stirring. The mixture was warmed to 70° C.
5) With high-speed mixing the oil phase of step 1 was added to the water phase of step 4. The emulsion was mixed to form uniform dispersion of the oil phase.
6) The Dapsone solution was added to step 5 with continued mixing. The emulsion was cooled.

### EXAMPLE 3

The following formulations were prepared:

| | % w/w | | |
|---|---|---|---|
| Excipient | 4-A | 4-B | 4-C |
| Dapsone | 2.0 | 1.0 | 1.0 |
| Isopropyl myristate | 30.0 | 20.0 | 20.0 |
| Octyldodecanol | — | — | 10.0 |
| Cetyl palmitate | — | 10.0 | 5.0 |
| Pemulen TR2 | 0.3 | 0.3 | 0.3 |
| Carbopol 980 | 0.2 | 0.4 | 0.2 |
| Propylene glycol | — | 10.0 | 10.0 |
| Ethoxydiglycol | 10.0 | — | — |
| Benzyl alcohol | — | — | 1.0 |
| Methylparaben | 0.15 | 0.15 | — |
| Butylated hydroxytoluene | 0.05 | 0.05 | 0.05 |
| Purified water | qs 100 | qs 100 | qs 100 |
| Sodium hydroxide, 10% | 0.5 | 0.5 | 0.5 |

Procedure:
1) The oil phase (isopropyl myristate, octyldodecanol, cetyl palmitate) was warmed to 60° C.
2) The Dapsone was added to step 1) and stirred to wet.
3) The Pemulen TR2 and Carbopol 980 were dispersed in the purified water with propeller mixing. The dispersion was warmed to 65° C.
4) The preservative (methylparaben or benzyl alcohol) and the BHT were added to the ethoxydiglycol or propylene glycol.
5) The step 4 solution was added to step 3) water phase.
6) With high-speed mixing the step 1) oil phase was added to the step 5) water phase.
7) The sodium hydroxide was added with mixing.
8) The cream was cooled to room temperature.

### EXAMPLE 4

The following formulation was prepared:

| Excipient | % w/w |
|---|---|
| Dapsone | 1.0 |
| White petrolatum | 5.0 |
| Isopropyl palmitate | 5.0 |
| Emulium Delta ® | 6.0 |
| Purified water | qs 100 |
| Carbopol 980 | 0.2 |
| Dimethyl isosorbide | 5.0 |
| Ethoxydiglycol | 5.0 |
| Methylparaben | 0.2 |
| Propylparaben | 0.05 |
| Sodium hydroxide solution, 10% | 0.2 |

The compounding procedure was the same as in Example 2. The dimethyl isosorbide was combined with the ethoxydiglycol to form the in-process solvent for Dapsone.

### EXAMPLE 5

The following formulation was prepared:

| Excipient | % w/w |
|---|---|
| Dapsone | 1.0 |
| Stearyl alcohol | 7.0 |
| Cetyl alcohol | 1.5 |
| Caprylic/capric triglyceride | 10.0 |
| Diisopropyl sebacate | 5.0 |
| Sorbitan monostearate | 2.0 |
| Polyoxyethylene 40 stearate | 2.5 |
| Purified water | Qs 100 |
| Dimethyl isosorbide | 10.0 |
| Polyethlene glycol 400 | 10.0 |
| Benzyl alcohol | 1.2 |
| Phosphate buffer | pH 7 |

Procedure:
1) The stearyl alcohol, cetyl alcohol, caprylic/capric triglyceride, diisopropyl sebecate, sorbitan monostearate, and polyoxyethylene 40 stearate were combined and melted at 70° C.
2) The dimethyl isosorbide, polyethylene glycol 400, and benzyl alcohol were combined. The Dapsone was added and wetted with mixing.
3) The purified water was combined with the phosphate buffer and warmed to 70° C.
4) With high-speed mixing the oil phase was added to the water phase.
5) The step 2) drug phase was added with mixing.
6) The cream was cooled to room temperature with mixing.

### EXAMPLE 6

The following formulation was prepared:

| Excipient | % w/w |
|---|---|
| Dapsone | 0.5 |
| Octyldodecanol | 10.0 |
| Cetostearyl alcohol | 5.0 |
| Purified water | Qs 100 |
| Pemulen TR2 | 0.3 |
| Carbomer 980 | 0.3 |
| Propylene glycol | 15.0 |
| Ethoxydiglycol | 20.0 |
| Benzyl Alcohol | 1.5 |
| Sodium hydroxide solution, 10% | 0.5 |

Procedure:
1) The octyldodecanol and cetostearyl alcohol were combined and warmed to melt at 70° C.
2) The propylene glycol, ethoxydiglycol, and benzyl alcohol were combined. The Dapsone was added and dissolved.
3) The purified water was warmed to 70° C.
4) The Pemulen TR2 and Carbopol 980 were added to step 1) oil phase and stirred to disperse.
5) With high-speed mixing the oil phase was added to the water phase.
6) The step 2) drug phase was added with continued mixing.
7) The sodium hydroxide solution was added and mixed.
8) The cream was cooled to room temperature.

### EXAMPLE 7

The following formulation was prepared:

| Excipient | % w/w |
|---|---|
| Dapsone | 2.0 |
| White petrolatum | 5.0 |
| Emulsifying Wax, NF | 10.0 |
| Isopropyl myristate | 10.0 |
| Diisopropyl adipate | 10.0 |
| Butylated hydroxytoluene | 0.05 |
| Propylene glycol | 10.0 |
| Benzyl alcohol | 1.5 |
| Purified water | qs 100 |

Procedure:
1) The white petrolatum, emulsifying wax, isopropyl myristate, diisopropyl adipate, and BHT were combined and melted at 70° C.
2) The Dapsone was added to the mixture of propylene glycol and benzyl alcohol and mixed to wet.
3) The purified water was warmed to 70° C.
4) With high-speed mixing the oil phase was added to the water.
5) The Dapsone solution was added with continued mixing.
6) The product was cooled to room temperature with mixing.

### EXAMPLE 8

The following formulation was prepared:

| Excipient | % w/w |
|---|---|
| Dapsone | 3.0 |
| Cetyl alcohol | 0.5 |
| Stearic acid | 7.0 |
| Mineral oil | 7.0 |
| PPG-2 myristyl ether propionate | 4.0 |
| Laureth 4 | 2.0 |
| Sodium laureth sulfate | 0.5 |
| Purified water | qs 100 |
| Propylene glycol | 15.0 |
| Ethoxydiglycol | 15.0 |
| Carbopol 980 | 0.4 |
| Triethanolamine | qs pH 6 |
| Methylparaben | 0.15 |
| Propylparaben | 0.03 |

General Procedure:
1) The cetyl alcohol, stearic acid, mineral oil, PPG-2 myristyl ether propionate, and laureth 4 were combined and melted to 70° C.
2) The Carbopol 980 was dispersed in the purified water with high-speed mixing.
3) The sodium laureth sulfate was added to the water phase, and the water phase was warmed to 70° C.
4) The parabens and the Dapsone were dissolved in the mixture of propylene glycol and ethoxydiglycol.
5) With high-speed mixing the oil phase was added to the water phase.
6) The Dapsone solution was added with continued mixing.
7) The triethanolamine was added for neutralization and the product was cooled to room temperature with mixing.

### EXAMPLE 9

The following formulations were prepared:

| | | % w/w | | | |
|---|---|---|---|---|---|
| Ingredient | Grade | A | B | C | D |
| Dapsone | USP | 5.0 | 2.0 | 5.0 | 5.0 |
| White Petrolatum | USP | 8.0 | 10.0 | — | 5.0 |
| Cetostearyl Alcohol | NF | 5.0 | 7.0 | 3.0 | — |
| Cetyl Palmitate | NF | — | — | 3.0 | 5.0 |
| Isopropyl Myristate | NF | 5.0 | 5.0 | 8.0 | 8.0 |
| Trilaureth-4 Phosphate | NA | 1.0 | 1.5 | — | — |
| Laureth-23 | NA | — | — | 3.0 | — |
| Ethoxydiglycol | NF | 23.0 | 10.0 | 25.0 | 20.0 |
| Carbomer Copolymer | NF | 0.15 | 0.15 | 0.20 | 0.20 |
| Carbomer 940 | NF | 0.15 | 0.15 | — | 0.20 |
| Methylparaben | NF | 0.20 | 0.20 | 0.2 | 0.20 |
| Propylparaben | NF | 0.04 | 0.04 | 0.04 | 0.04 |
| Sodium Hydroxide | NF | pH 5-7 | pH 5-7 | pH 5-7 | PH 5-7 |
| Purified Water | USP | To 100 | To 100 | To 100 | To 100 |

General Procedure:
1) Combine Oil Phase ingredients in vessel and warm to 65° C. to 75° C. to melt. Oil Phase ingredients include white petrolatum, cetostearyl alcohol, cetyl palmitate, isopropyl myristate, and laureth-23.
2) Prepare Active Phase in separate vessel by adding methylparaben, propylparaben, and dapsone to ethoxydiglycol and stirring to dissolve.
3) Prepare Water Phase by adding carbomer to purified water with high-speed mixing to form uniform dispersion. Warm resulting dispersion to 65° C. to 75° C.
4) Add trilaureth-4 phosphate when present to Water Phase.
5) Compound emulsion by adding Oil Phase to Water Phase with high-speed mixing.
6) Slowly add Active Phase to emulsion with continued high-speed mixing.
7) Add sodium hydroxide (as aqueous solution) to emulsion and mix to combine.
8) Cool emulsion with stirring to room temperature.

### EXAMPLE 10

The following formulations were prepared:

| | % w/w | | |
|---|---|---|---|
| Ingredient | A | B | |
| Dapsone, USP | 3.0 | 2.0 | 3.0 |
| Propylene glycol, USP | 20.0 | — | — |
| Ethoxydiglycol, NF | — | 10.0 | 10.0 |
| Dimethyl Isosorbide | — | — | 5.0 |
| Polyethylene Glycol 400, NF | 44.0 | 55.0 | 51.0 |
| Polyethylene Glycol 3350, NF | 25.0 | 25.0 | 20.0 |
| Stearyl Alcohol, NF | 5.0 | 5.0 | 8.0 |
| Arlacel 165¹ | 3.0 | 3.0 | 3.0 |

¹Arlacel 165 is a tradename mixture of glyceryl stearate and POE 100 stearate. It is available from ICI Surfactants.

General Procedure:
1) For Active Phase, add the Dapsone to either the propylene glycol or the ethoxdiglycol and mix to combine and wet.
2) For Base, combine the remaining ingredients and warm to 55° C. to 60° C. to melt completely.
3) With mixing, add the Active Phase to the Base. Stir to uniformly combine.
4) Cool the mixture with continued mixing. Cool to room temperature.

### REFERENCES

| | | |
|---|---|---|
| a) | Barabas. | U.S. Pat. No. 4,853,439 |
| b) | Cho, et al. | U.S. Pat. No. 4,920,145 |
| c) | Vichroski, et al. | U.S. Pat. No. 5,437,867 |
| d) | Kompis, et al. | U.S. Pat. No. 5,721,242 |
| e) | Osborne. | U.S. Pat. No. 5,863,560 |
| f) | Preuilh, et al. | U.S. Pat. No. 6,106,848 |
| g) | Castro, et al. | U.S. Pat. Nos. 6,113,888; |
| | | 6,214,322 |
| h) | Fischetti, et al. | U.S. Pat. Nos. 6,056,955; |
| | | 6,277,399; 6,248,324; 6,432,444 |
| i) | Stroud, et al. | U.S. Pat. No. 6,231,837 |

The entire disclosures of all patents, patent applications, publications and references cited in this specification are incorporated herein by reference as if fully reproduced in this specification.

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. An emulsive composition comprising the components a) Dapsone or its derivative, b) a solvation medium, c) an emulsifier system and d) an oil phase component.

2. An emulsive composition according to claim 1 further comprising water as component e).

3. An emulsive composition according to claim 2 wherein the water and the solvation medium at least partially dissolve the Dapsone or its derivative.

4. An emulsive composition according to claim 1 wherein the weight percentages of components relative to the total weight of the emulsive composition comprise:
a) Dapsone or its derivative from about 0.005 percent to about 30 percent;
b) solvation medium from about 0.5 percent to about 99 percent;
c) emulsifier system from about 0.1 percent to about 30 percent;
d) oil phase component from about 0.1 weight percent to about 75 percent.

5. An emulsive composition according to claim 2 wherein the water weight percentage ranges up to about 99 percent.

6. An emulsive composition according to claim 4 further including water in a weight percentage range of up to about 99 percent.

7. An emulsive composition according to claim 1 wherein the weight percentages of components relative to the total weight of the emulsive composition comprise:
a) Dapsone or its derivative from about 0.1 percent to about 25 percent;
b) solvation medium from about 0.5 percent to about 50 percent;
c) emulsifier system from about 0.5 percent to about 25 percent;
d) oil phase component from about 0.1 weight percent to about 50 percent.

8. An emulsive composition according to claim 1 further comprising optional water, and wherein the weight percentages of components relative to the total weight of the emulsive composition are selected from among the following ranges such that the total amounts equal 100 percent, and each succeeding range for each component is preferred relative to the preceding range:
a) Dapsone range selections are from about 0.005 percent to about 30 percent: about 0.1 percent to about 25 percent; about 0.1 percent to about 15 percent; about 0.1 percent to about 10 percent; about 0.2 percent to about 8 percent; about 0.5 to about 5 percent by weight of the emulsive 5 composition;
b) the solvation medium range selections are from about 0.5 percent to about 99 percent; about 0.5 percent to about 50 percent; about 5 percent to about 40 percent; about 5 percent to about 35 percent; about 5 percent to about 30 percent;
c) the emulsifier system range selections are from about 0.1 percent to about 30 percent; about 0.5 percent to about 25 percent; about 1 percent to about 25 percent; about 5 percent to about 25 percent; about 5 percent to about 20 percent;
d) the oil phase component range selections are from about 0.1 weight percent to about 75 percent; about 0.1 to about 50 percent; about 1 to about 45 percent; about 2 to about 40 percent; and
e) optional water range selections are from 0 percent to about 99 percent; from 0 to about 50 percent; from 0 to about 40 percent; from 0 to about 35 percent.

9. An emulsive composition according to claim 1 wherein the emulsifier system comprises a combination of a fatty alcohol and a surfactant.

10. An emulsive composition according to claim 9 wherein the surfactant comprises a nonionic or anionic surfactant or a combination thereof.

11. An emulsive composition according to claim 9 wherein the fatty alcohol comprises a C8 to C30 alcohol optionally substituted by additional hydroxyl groups, alkoxy groups of C1 to C6 carbons, or alkoxycarbonyl groups of 2 to 6 carbons, or alkyl amido groups of 2 to 6 carbons or any combination thereof.

12. An emulsive composition according to claim 1 wherein the solvation medium comprises an organic solvent in which Dapsone or its derivative is soluble.

13. An emulsive composition according to claim 12 wherein the organic solvent comprises a glycol, a polyol, a glycol or polyol ether or any combination thereof.

14. An emulsive composition according to claim 1 wherein the Dapsone is fully or partially dissolved in the solvation medium.

15. An emulsive composition according to claim 1 wherein the Dapsone is fully or partially dissolved in the solvation medium and the oil phase component.

16. An emulsive composition according to claim 1 wherein the emulsifier system comprises a carbomer copolymer.

17. An emulsive composition comprising a) Dapsone, b) a glycol ether, c) an emulsifier system, d) an oil phase component, and e) optional water.

18. An emulsive composition of claim 17 wherein the glycol ether is ethoxydiglycol.

19. An emulsive composition of claim 18 including a carbomer copolymer.

20. An emulsive composition of claim 18 wherein the emulsifier system comprises a fatty alcohol and a surfactant.

21. An emulsive composition of claim 17 wherein the Dapsone is at a concentration from about 2% to about 5% by weight of the composition.

22. An emulsive composition of claim 17 wherein the ratio of the concentration of the Dapsone to the concentration of the glycol ether is such that the Dapsone is soluble in the glycol ether.

23. An emulsive composition according to any of the preceding claims wherein acidic and/or basic components are neutralized or buffered to a pH of from about 4 to 8, preferably 5 to 8, especially preferably 5 to 7.

24. An emulsive composition comprising: Dapsone, wax or petrolatum, C10 to C20 alcohol, C10 to C20 alkyl C10 to C20 alkanoate, C2 to C6 alkyl (C10 to C20) alkanoate, phosphate surfactant, ethoxydiglycol, polyacrylic acid or copolymer thereof, preservative, optional neutralizing agent and optional water.

25. An emulsive composition according to claim 24 having component ranges according to claim 8.

26. An emulsive composition according to claim 24 wherein the ranges are Dapsone—2 to 5%, wax or petrolatum—5 to 10%, C10 to C20 alcohol—0 to 5%, C10 to C20 alkyl (C10 to C20) alkanoate—0 to 5%, C2 to C6 alkyl (C10 to C20 alkanoate—5 to 8%, phosphate surfactant—0 to 5%, ethoxydiglycol—10 to 30%, polyacrylic acid or copolymer thereof—0.1 to 0.2%, preservative 0.04 to 0.2%, neutralizing agent and optional water to 100%, the percentages being by weight relative to the total weight of the composition.

27. An emulsive composition according to claim 24 comprising Dapsone, wax or petrolatum, cetostearyl alcohol, cetyl palmitate, isopropyl myristate, trilaureth-4 phosphate, ethoxydiglycol, carbomer copolymer, carbomer 940, preservative, optional neutralizing agent and optional water.
